(19) United States
(12) Patent Application Publication
(10) Pub. No.: US 2022/0378705 A1
(43) Pub. Date: Dec. 1, 2022

(54) METHOD FOR PREPARING PHARMACEUTICAL COMPOSITIONS CONTAINING AMPHIPHILIC ACTIVE INGREDIENTS

(71) Applicant: PHARMADEV SA, Geneve 1 (CH)

(72) Inventors: François FAURAN, Coppet (CH); Didier MULLER, Veyre-Monton (FR); Jean-Luc BARNOUX, Chaon-Montperreux (FR)

(21) Appl. No.: 17/614,076

(22) PCT Filed: May 23, 2020
(86) PCT No.: PCT/EP2020/064345

§ 371 Date: Jul. 11, 2022

(30) Foreign Application Priority Data
May 24, 2019 (FR) FR1905539

(51) Int. Cl. A61K 9/16; A61K 31/192; A61K 9/28; A61K 9/20; A61K 31/522; A61K 31/137

(52) U.S. Cl. CPC A61K 9/1694; A61K 31/192; A61K 9/1635; A61K 9/1623; A61K 9/1611; A61K 9/1652; A61K 9/1617; A61K 9/28; A61K 9/2018; A61K 9/2013; A61K 9/2027; A61K 31/522; A61K 31/137

(57) ABSTRACT

The present invention relates to a method for granulating an amphiphilic active ingredient or a pharmaceutically acceptable salt thereof, comprising a step for coating the active ingredient in a polar aprotic solvent in the presence of a polymer binder to obtain a granule.

### FIELD OF THE INVENTION

The present invention relates to a method for preparing active ingredient granules entering into the preparation of pharmaceutical compositions, and to the granules and pharmaceutical compositions themselves.

### TECHNICAL BACKGROUND

Amphiphilic active ingredients are characterized by a lipophilic hydrocarbon skeleton and one or more ionizable, generally weak acid or base functions imparting the hydrophilic character, which can be amplified by salification with a pharmaceutically acceptable anion or cation.

Among these amphiphilic active ingredients are nonsteroidal anti-inflammatory drugs (NSAIDs), including anthranilic, arylacetic, and arylpropionic (or propanoic) acids substituted in position 2 or 3.

These active ingredients are thus characterized by a weak carboxylic acid function, constituting the hydrophilic pole, bonded:
either directly (salicylates and anthranilic acids),
or by a carbon atom (arylacetic acids),
or by two carbon atoms (aryl-2- or -3-propionic acids), to a substituted aromatic or heteroaromatic entity constituting the hydrophobic pole.

These active ingredients are primarily used as analgesics in the treatment of mild to moderate pain linked to dysmenorrhea, headaches, migraine, postoperative and dental pain, and as anti-inflammatories in the treatment of spondyloarthritis, osteoarthritis, rheumatoid arthritis, and soft tissue disorders.

These active ingredients, when they are intended for orally administrable compositions, are generally subjected to a wet granulation particularly adapted to the amphiphilic character of NSAIDs. However, the granules thus obtained pose a certain number of technological problems during the formulation phase, particularly in tablet form, such as problems of poor flowability, sticking, binding, or capping.

To overcome this problem, it has mainly been proposed to use surface-treated compression punches. Thus, Robert et al. (2003) demonstrated the advantage of using chromium- or chromium nitride-treated punches to prevent sticking during the compression of ibuprofen granules obtained by wet granulation. Similarly, Al-Karawy & Leopold (2018) tested the adhesive properties of ibuprofen and its sodium salt in four punches with different coatings: titanium nitride, chromium nitride, diamond-like carbon, and hard chromium.

However, the dissolution rate of the active ingredient with this type of compression is not optimal and alternative solutions are still necessary.

### SUMMARY OF THE INVENTION

The present invention follows from the unexpected discovery by the inventors that a granulation of an amphiphilic active ingredient in a polar aprotic solvent medium with a coating polymer made it possible to prevent interactions resulting from the amphiphilic structure of these active ingredients, particularly the sticking phenomenon. In an advantageous and unexpected way, this method of granulation also makes it possible to optimize the dissolution rate of the active ingredient.

Thus, the present invention relates to a method for granulating an amphiphilic active ingredient or a pharmaceutically acceptable salt thereof, comprising a step for coating the active ingredient in a polar aprotic solvent in the presence of a polymer binder so as to obtain a granule.

In one embodiment of the invention, the above-defined method also comprises a step of drying and/or sieving the granule.

The present invention also relates to a granule that can be obtained by the above-defined method.

The present invention also relates to a granule comprising a polar active ingredient or a pharmaceutically acceptable salt thereof, coated with a polymer binder and having a density of 0.5 to 0.7 g/mL and/or a flow rate of 3 to 15 g/sec.

The present invention also relates to a pharmaceutical composition or medicine comprising a granule as defined above.

In one embodiment of the invention, the pharmaceutical composition or medicine as defined above also comprises at least one other active ingredient, preferably an analgesic and/or an antihistamine.

In another embodiment of the invention, the pharmaceutical composition or medicine as defined above is for use in the prevention or treatment of pain, fever, and/or inflammation.

### DETAILED DESCRIPTION OF THE INVENTION

### Amphiphilic Active Ingredient

Preferably, the amphiphilic active ingredient according to the invention comprises at least one carboxylic acid group and at least one aryl group comprising from 6 to 50 carbon atoms.

Preferably, the amphiphilic active ingredient is a nonsteroidal anti-inflammatory drug (NSAID).

As understood herein, amphiphilic, also known as bipolar, active ingredients of the NSAID type are molecules which hydrophobic character is characterized by their octanol-water partition coefficient (Log Po/w), preferably comprised between 2 and 5), and which hydrophilic character is characterized by their weak carboxylic acid function (preferably with a pKa on the order of 4). These amphiphilic structures develop a measurable surface tension in solution.

Preferably, the amphiphilic active ingredient is an arylacetic NSAID, an arylproprionic NSAID, or an anthranilic NSAID.

Preferably, the amphiphilic active ingredient is selected from the group consisting of ibuprofen, ketoprofen, naproxen, flurbiprofen, oxaprozin, ibufenac, diclofenac, aceclofenac, sulindac, etodolac, ketorolac, indomethacin, mefenamic acid, and niflumic acid.

TABLE 1

| | | Log | |
|---|---|---|---|
| | | P_{o/w} | pKa |
| Anthranilic Acids | | | |
| NIFLUMIC ACID | 2-[3-(trifluoromethyl(anilino]pyridine-3- | 4.43 | 4.44 |
| | carboxylic acid | | |
| MEFENAMIC | 2-(2,3-dimethylanilino)benzoic acid | 4.20 | 4.20 |
| ACID | | | |
| Arylacetic Acids | | | |
| DICLOFENAC | 2-[2-(2,6-dichloroanilino)phenyl]acetic acid | 4.51 | 4.15 |
| SULINDAC | 2-[(3Z)-6-fluoro-2-methyl-3-[(4- | 3.42 | 4.70 |
| | methylsulfinylphenyl)hoursmethylidene]inden-1- | | |
| | yl]acetic acid | | |
| INDOMETHACIN | 2-[1-(4-chlorobenzoyl)-5-methoxy-2-methylindol- | 4.27 | 4.50 |
| | 3-yl]acetic acid | | |
| ETODOLAC | 2-(1,8-diethyl-4,9-dihydro-3H-pyrano[3,4-b]indol- | 2.5 | 4.65 |
| | 1-yl)acetic acid | | |
| KETOROLAC | 2-amino-2-(hydroxymethyl)propane-1,3-2- | 2.3 | 3.84 |
| | amino-2-(hydroxymethyl)-1,3-propanediol(±)-5- | | |
| | benzoyl-2,3-dihydro-1H-pyrrolizine-1-carboxylic | | |
| | acid diol(±)-5-benzoyl-2,3-dihydro-1H-pyrrolizine- | | |
| | 1-carboxylic acid* | | |
| Arylpropanoic Acids | | | |
| IBUPROFEN | 2-(4-Isobutylphenyl)propanoic acid* | 3.87 | 4.91 |
| KETOPROFEN | 2-(3-Benzoylphenyl)propanoic acid* | 3.12 | 4.45 |
| NAPROXEN | 2-(6-methoxynaphthalen-2-yl)propanoic acid* | 3.18 | 4.15 |
| FLURBIPROFEN | 2-(3-fluoro-4-phenylphenyl)propanoic acid | 4.16 | 4.17 |
| OXAPROZIN | 3-(4,5-diphenyl-1,3-oxazol-2-yl)propanoic acid | 4.19 | 4.30 |

*optical isomers

Preferably, the pharmaceutically acceptable salt of the amphiphilic active ingredient is selected from the group consisting of a lithium salt, a sodium salt, a potassium salt, a calcium salt, an aluminum salt, a magnesium salt, a zinc salt, an arginine salt, a lysine salt, a histidine salt, a choline salt, an ethanolamine salt, a diethanolamine salt, a triethanolamine salt, an ethylenediamine salt, and a meglumine salt.

Preferably, the amphiphilic active ingredient or the pharmaceutically acceptable salt thereof is the lysine salt or the sodium salt of ibuprofen.

In the process of the invention, it is preferred that the amphiphilic active ingredient or pharmaceutically acceptable salt thereof is in crystalline form.

Preferably, the method according to the invention is thus a crystal coating method, particularly a method for coating crystals of an amphiphilic active ingredient or a pharmaceutically acceptable salt thereof.

### Polymer Binder

Preferably, the polymer binder is a polyvinylpyrrolidone (povidone) or a polyvinylpyrrolidone copolymer, particularly copovidone (a copolymer of polyvinylpyrrolidone and vinyl acetate), a polyethylene glycol (PEG), a polyoxypropylene copolymer (particularly of the POLOXAMER® type), or a methacrylate copolymer (particularly of the EUDRAGIT® type).

Preferably, the quantity of polymer binder is of at least 5% by weight relative to the weight of active ingredient.

### Polar Aprotic Solvent

As understood herein, a polar aprotic solvent has a dielectric constant significantly lower than that of water for a higher or comparable dipole moment.

Preferably, and as will be clear to a person skilled in the art, the polar aprotic solvent according to the invention is a pharmaceutically acceptable solvent, particularly a class 2 or 3 solvent according to the classification of the European Pharmacopoeia.

Preferably, the polar aprotic solvent according to the invention is selected from the group consisting of acetone, ethyl acetate, acetonitrile, and N,N-dimethylformamide.

TABLE 2

| | Dielectric | Dipole moment | | |
|---|---|---|---|---|
| Solvent | constant ε | μ(D) | Solubility | Class |
| Water | 80 | 1.85 | −3.5 | N.A. |
| Ethyl acetate | 6 | 1.78 | −7.4 | 3 |
| Acetone | 21 | 2.88 | −7.9 | 3 |
| Acetonitrile | 37 | 3.92 | N.D. | 2 |
| N,N-dimethylformamide | 38 | 3.82 | −6.8 | 2 |

N.A.: not applicable/
N.D.: not determined
Solubility: Log₁₀ of the molar fraction of the ibuprofen sodium salt dissolved in the solvent (according to Bustamante et al. (2000) International Journal of Pharmaceutics 194: 117-124, which is incorporated herein by reference).

In particular, acetonitrile, N,N-dimethylformamide, acetone, and ethyl acetate essentially do not solubilize ibuprofen sodium salt dihydrate, which means that 1 g of the active ingredient in question is not dissolved by 10,000 mL of solvent, making it possible to facilitate the granulation.

Thus, it is preferred in the method of the invention that the amphiphilic active ingredient or the pharmaceutically acceptable salt thereof is essentially not solubilized, or essentially not dissolved, in the polar aprotic solvent according to the invention, which means that the Logio of the molar fraction of the amphiphilic active ingredient or of the pharmaceutically acceptable salt thereof dissolved in the solvent is preferably less than −5, more preferably less than −6, under standard temperature and pressure conditions. In contrast, it is preferred in the method of the invention that the polymer binder is soluble, or dissolved, in the polar aprotic solvent according to the invention.

### Granule

As is clear to the person skilled in the art, the granule according to the invention is preferably a granule coated with the polymer binder, in which the amphiphilic active ingredient or pharmaceutically acceptable salt thereof is in crystalline form. In other words, the granule according to the invention preferably comprises, or consists of, at least one crystal of amphiphilic active ingredient according to the invention or of a pharmaceutically acceptable salt thereof that is coated with the polymer binder according to the invention.

Preferably, the granule according to the invention essentially consists of the amphiphilic active ingredient or a pharmaceutically acceptable salt thereof, and of the polymer binder, which means that the granule according to the invention comprises the amphiphilic active ingredient or a pharmaceutically acceptable salt thereof, the polymer binder, and possibly water and/or impurities.

A granule in which the quantity of polymer binder is of at least 5% by weight relative to the weight of active ingredient or pharmaceutically acceptable salt thereof is preferred according to the invention.

More preferably, the coating according to the invention is performed by mixing the amphiphilic active ingredient or a pharmaceutically acceptable salt thereof with the polymer binder in a ratio by weight of between 10 and 20% of binder relative to the weight of the active ingredient or the pharmaceutically acceptable salt thereof, in particular for a volume of solvent between 15 and 35% relative to the weight of the active ingredient or the pharmaceutically acceptable salt thereof.

The coating can be performed in a mixer granulator capable of operating under reduced pressure and inert atmosphere.

The pressure difference between the mixer and the outside can be used to introduce the solvent and perform the drying of the granule obtained. For less volatile solvents, it is possible to substantially reduce the differential pressure and operate at up to 30 mBar.

After the optional drying step, the granule obtained can be sieved, particularly through two sieves in which the nominal dimensions of the meshes are respectively 1.5 mm and 1 mm.

Advantageously, the method according to the invention makes it possible to eliminate the crystals of amphiphilic active ingredient according to the invention, or of a pharmaceutically acceptable salt thereof, which size is less than 75 μm, in particular less than 50 μm, or to reduce their quantity.

Preferably, the size of a granule according to the invention, which comprises at least one crystal of amphiphilic active ingredient according to the invention, or of a pharmaceutically acceptable salt thereof, is of about 1.3 to 1.7 times, preferably of about 1.4 to 1.6 times, even more preferably of about 1.5 times, the size of the crystal of amphiphilic active ingredient according to the invention, or of the pharmaceutically acceptable salt thereof. Preferably, the size is measured as the largest dimension of the granule or crystal.

Advantageously, the method according to the invention eliminates the need for the conventional operations of milling and/or calibration.

Preferably, the method according to the invention does not include a step of drying by fluidized bed, by pulverization, or by atomization. Preferably also, the method according to the invention does not include a step of precipitating the amphiphilic active ingredient, or a pharmaceutically acceptable salt thereof, and/or the polymer binder.

Preferably, the granules obtained have a density on the order of 0.50 to 0.70 g/mL and a flow rate on the order of 3 to 15 g/sec.

### Pharmaceutical Composition or Medicine

Preferably, the pharmaceutical composition or medicine according to the invention is intended for or in a form suitable for oral administration.

Preferably, the pharmaceutical composition or medicine according to the invention is presented in the form of a tablet, a capsule, or granules for a drinkable suspension.

Preferably, the pharmaceutical composition or medicine according to the invention is fast-dissolving.

Preferably, the pharmaceutical composition or medicine according to the invention also comprises at least one pharmaceutically acceptable vehicle or excipient.

Preferably, the pharmaceutical composition or medicine according to the invention is in the form of a tablet or a capsule comprising at least one hydrophilic excipient, preferably silica and mannitol, at least one disintegrating agent, preferably croscarmellose sodium, and at least one lubricating agent, preferably magnesium stearate.

An external phase is added to the granules according to the invention in order to form a tablet or a capsule. Thus, the above excipients and agents constitute the external phase of the tablet or capsule. The external phase more particularly comprises agents selected from:
filling agents, such as mannitol or PROSOLV HD90® (a composition comprising 98% microcrystalline cellulose and 2% colloidal silica);
disintegrating agents such as croscarmellose sodium;
flow agents such as hydrated colloidal silica, in particular according to the European Pharmacopoeia (particularly precipitated silica of the LEVILITE® type or mesoporous silica of the SYLOID® type);
lubricating agents such as magnesium stearate.

Advantageously, the granules according to the invention are such that it is not always necessary to add a lubricant to the external phase in order to prepare a tablet or a capsule.

Thus, the pharmaceutical compositions and medicines according to the invention, particularly in tablet or capsule form, preferably do not comprise a lubricating agent.

Preferably, the pharmaceutical compositions and medicines according to the invention, in particular the tablets according to the invention, do not comprise excipients that can modify the gastric pH and generate a release of carbonic gas in the stomach. More preferably, the pharmaceutical compositions and medicines according to the invention, in particular the tablets according to the invention, do not comprise sodium carbonates and bicarbonates, sodium citrates and phosphates, or strong bases such as potash.

Preferably, the external phase represents less than 50% of the weight of the mixture of the granule and the external phase.

The compression of the tablet can be performed in a rotary machine with conventional punches.

The coating of the tablet can be performed conventionally in a film-coating machine. Preferably, the coating of the tablet will be conducted at a temperature below 35° C. The coating agents of the tablet are such that they adhere to the surface of the tablets taking into account the surface tension of the ibuprofen sodium salt. According to the invention, polyvinyl alcohol-based coatings are preferred over those that are hypromellose-based.

### Other Active Ingredient

Preferably, the other active ingredient is selected from the group consisting of paracetamol, codeine, oxycodone, caffeine, phenylephrine, and pseudoephedrine.

The invention will be further explained, in a non-limiting way, by the examples and figures below.

### DESCRIPTION OF THE FIGURES

FIG. 1 is a flow chart for preparing a tablet according to the invention.

FIG. 2 represents the in vitro dissolution kinetics of tablets according to the invention (G17, G34-F42, G35-F46) and commercial tablets (NUROFEN® FLASH 512 mg batch BW538, NUROFEN® 400 mg batch DL365) (percentage of dissolution, y-axis) under the same conditions (pH=6.8) as a function of time (x-axis, in minutes).

FIG. 3 represents the percentage of dissolution of a tablet according to the invention (IBUNA 512 mg) and of the product NUROFEN® 400 mg batch DL 365 (y-axis, in μg/mL) as a function of time (x-axis, in minutes) in 900 mL of a solution at pH 6.8, under agitation at 50 rpm.

FIG. 4 represents the dissolution kinetics of a tablet according to the invention at pH=6.8 in vitro (percentage of dissolution) and in vivo (as a percentage of Cₘₐₓ) (y-axis) as a function of time (x-axis, in minutes).

FIG. 5 represents images obtained by scanning electron microscopy (magnification 10 kV×100) of crystals of ibuprofen sodium salt dihydrate (on the left) and of granules according to the invention obtained from these crystals by coating in a polymer binder (on the right).

FIG. 6 represents, from top to bottom, the X-ray powder diffraction (XRPD) spectra of ibuprofen sodium salt dihydrate:
(i) in crystalline form,
(ii) of a film-coated tablet according to the invention,
(iii) of a granule according to the invention obtained from copovidone (polymer binder) and acetone (polar aprotic solvent),
(iv) of a granule according to the invention obtained from copovidone (polymer binder) and acetonitrile (polar aprotic solvent).

The x-axis represents the value of the 2-Theta angle in degrees and the y-axis represents the intensity of the diffracted rays.

### EXAMPLES

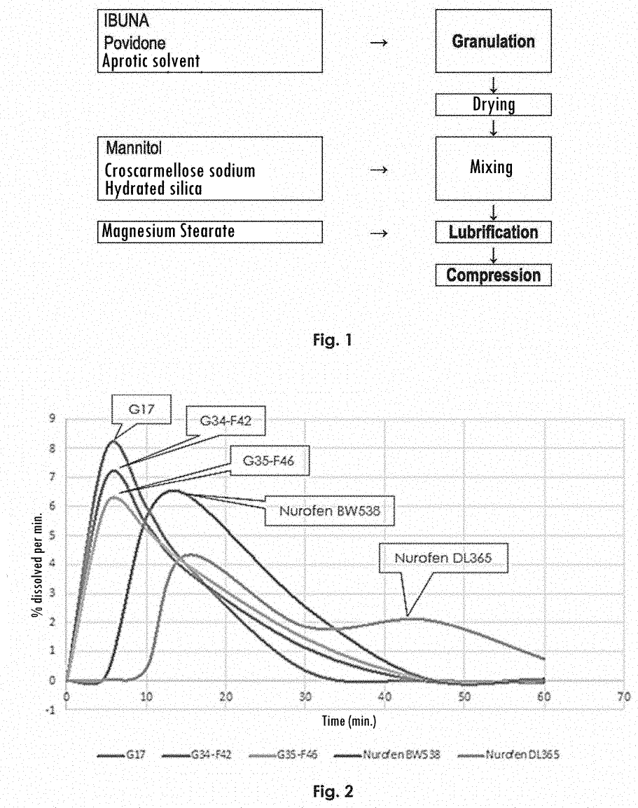

The general flow chart of the method used in the following examples is given in FIG. 1.

The compression is performed in a rotary machine of the KYLLIAN LX20 type with conventional punches. The coating of the tablet is performed in a film-coating machine of the DRIACOATER type (DRIAM GmbH).

### Examples 1, 2 and 3: Tablets of Ibuprofen Sodium Salt Obtained by Granulation in a Polar Aprotic Solvent

TABLE 3
EXAMPLE 1 - G17

| Ingredients | mg/tablet |
|---|---|
| Internal Phase | |
| Ibuprofen sodium salt dihydrate | 512.00 |
| (acetonitrile granulation) | |
| Povidone (PLASDONE K29/32) | 55.00 |
| Acetonitrile (intermediate solvent) | |
| External phase | |
| Mannitol (PEARLITOL ® SD200) | 235.55 |
| Croscarmellose sodium (VIVASOL ®) | 45.00 |
| Hydrated Silica (LEVILITE ®) | 100.00 |
| Magnesium stearate | 14.45 |
| Mixture before compression | |
| Flow time (Sec.) | 12.62 |
| Apparent volumes (mL) | |
| V10 | 269 |
| V1250 | 257 |
| Density (g/mL) | 0.34 |
| Compressibility index | 13 |
| Hausner ratio | 1.15 |
| Tablets | |
| Average mass (mg) | 962 |
| Hardness (N) | 75.7 |
| Disaggregation (min.) | |
| Dissolution % 15 min. | 90 |
| Friability % | 0.49 |
| Capping | 0 |
| Binding | 0 |

TABLE 4
EXAMPLE 2 - G34-F42

| Ingredients | mg/tablet |
|---|---|
| Internal Phase | |
| Ibuprofen sodium salt dihydrate | 512.00 |
| (acetone granulation) | |
| Povidone (PLASDONE K29/32) | 80.00 |
| Acetonitrile (intermediate solvent) | |
| External phase | |
| Mannitol (PEARLITOL ® SD200) | 235.55 |
| Croscarmellose sodium (VIVASOL ®) | 45.00 |
| Hydrated Silica (LEVILITE ®) | 100.00 |
| Magnesium stearate | 14.45 |
| Mixture before compression | |
| Flow time (g/sec.) | 13.72 |
| Apparent volumes (mL) | |
| V10 | 224 |
| V1250 | 211 |
| Density (g/mL) | 0.42 |
| Compressibility index | 13 |
| Hausner ratio | 1.14 |
| Tablets | |
| Average mass (mg) | 987 |
| Hardness (N) | 82.4 |
| Disaggregation (min.) | 10.4 |
| Dissolution % 15 min. | 81 |
| Friability % | 0.76 |
| Capping | 0 |
| Binding | 0 |

TABLE 5
EXAMPLE 3 - G35-F46

| Ingredients | mg/tablet |
|---|---|
| Internal Phase | |
| Ibuprofen sodium salt dihydrate | 512.00 |
| (ethyl acetate granulation) | |
| Povidone (PLASDONE K29/32) | 80.00 |
| Acetonitrile (intermediate solvent) | |
| External phase | |
| Mannitol (PEARLITOL ® SD200) | 242.62 |
| Croscarmellose sodium (VIVASOL ®) | 46.35 |
| Hydrated Silica (LEVILITE ®) | 46.00 |
| Magnesium stearate | 17.00 |
| Mixture before compression | |
| Flow time (g/sec.) | 15.27 |
| Apparent volumes (mL) | |
| V10 | 225 |
| V1250 | 212 |
| Density (g/L) | 0.37 |
| Compressibility index | 13 |
| Hausner ratio | 1.14 |
| Tablets | |
| Average mass (mg) | 970 |
| Hardness (N) | 80 |
| Disaggregation (min.) | 12.36 |
| Dissolution % 15 min. | 76.4 |
| Friability % | 0.2 |
| Capping | 0 |
| Binding | 0 |

The three examples reported above use different polar aprotic solvents according to the invention: acetonitrile in Example 1, acetone in Example 2, ethyl acetate in Example 3. Granulation in all 3 examples is performed with Povidone (Ph. Eur.).

The tables below present exemplary characteristics of granules according to the invention obtained with acetone:

TABLE 6
Granule batch 729740

| Ingredients | mg |
|---|---|
| Ibuprofen sodium salt dihydrate | 512.00 |
| Povidone (PLASDONE K29/32) | 55.00 |
| Acetone (intermediate solvent) | Quantum satis |
| Pharmacotechnical properties | |
| Flow time (Sec.) | 10.3 |
| Apparent volumes (mL) | |
| V10 | 182 |
| V1250 | 161 |
| Density before compaction (g/mL) | 0.55 |
| Density after compaction (g/mL) | 0.62 |
| Compressibility index | 21 |
| Hausner ratio | 1.13 |

TABLE 7
Granule batch 729650

| Ingredients | mg |
|---|---|
| Ibuprofen sodium salt dihydrate | 512.00 |
| Povidone (PLASDONE K29/32) | 55.00 |
| Acetone (intermediate solvent) | Quantum satis |
| Pharmacotechnical properties | |
| Flow time (Sec.) | 12.1 |
| Apparent volumes (mL) | |
| V10 | 185 |
| V1250 | 149 |
| Density before compaction (g/mL) | 0.54 |
| Density after compaction (g/mL) | 0.67 |
| Compressibility index | 36 |
| Hausner ratio | 1.24 |

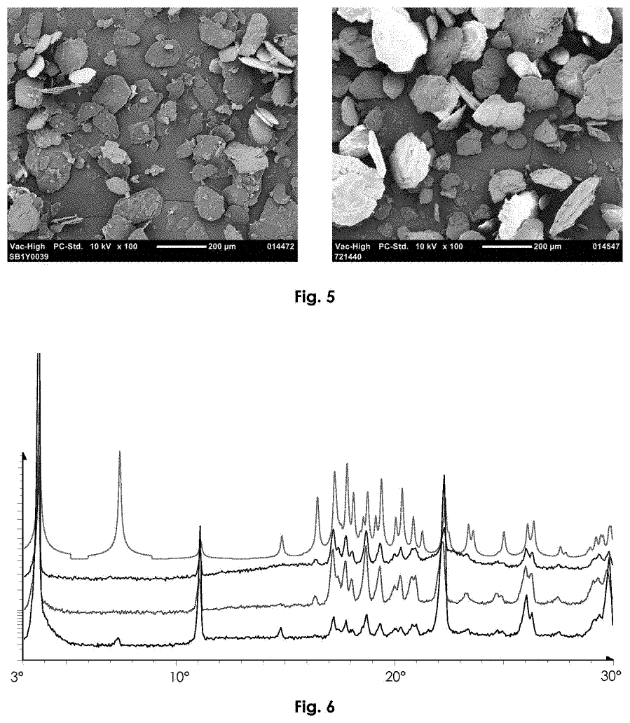

FIG. 5 shows scanning electron microscopy images of crystals of ibuprofen sodium salt dihydrate and of granules according to the invention obtained from these crystals by coating with a polymer binder. Due to the coating with the polymer binder, an increase in the size of the granules relative to the crystals, a reduction in the number of crystals of small size (less than 50 μm), and the maintenance of the crystalline form in the granules can be observed.

The external phases of the tablets comprise:
two hydrophilic excipients: Mannitol and Hydrated Silica (Ph. Eur.), the latter of which, depending on the type (LEVILITE® or SYLOID®), can have a more or less pronounced hydrophilic character;
a disintegrating agent: croscarmellose sodium, and
a lubricant: magnesium stearate.

These formulations make it possible to obtain a grain, composed of the active ingredient granule and of the external phase, that has good flowability, and suitable compressibility (HAUSNER ratio of 1.14-1.15) as evidenced by tablets which hardness is between and 76 and 83 N, and that shows no capping, no trace of sticking, and no binding, with a friability of less than 1%.

Moreover, the test of their dissolution in accordance with the European Pharmacopoeia at 37° C. in 900 mL of buffer at pH 6.8 reveals a fast dissolution of the ibuprofen sodium salt, since the following are dissolved within 15 minutes:
90% of Example 1 (acetonitrile granulation)
80% of Example 2 (acetone granulation)
76% of Example 3 (ethyl acetate granulation).

These results are confirmed by comparing the dissolution rates, expressed as a percentage dissolved per minute of these three formulations according to the invention as a function of time relative to that of tablets of reference products formulated either with ibuprofen sodium salt, i.e. NUROFEN® MAX STRENGTH 512 mg batch BW 538, or with ibuprofen in acid form, i.e. NUROFEN® 400 mg batch DL365.

The results of FIG. 2 show that, advantageously, the formulations according to the invention reach their maximum rate from the 5th minute, unlike the two reference products, which dissolution does not start until after 5 minutes (NUROFEN® MAX STRENGTH 512 mg) and 10 minutes (NUROFEN® 400 mg), their maximum rates not being reached until after 12 to 15 minutes, respectively, with higher maximum rate values for the formulations using the salified form of ibuprofen.

### Example 4: Film-Coated Tablets of Ibuprofen Sodium Salt Obtained by Granulation in a Polar Aprotic Solvent

The formulation below resulted from Example 2 above. After addition of the external phase and compression to form the tablet, it is finished with a polyvinyl alcohol-based film-forming composition:

TABLE 8

| | | 512 mg/ |
|---|---|---|
| Ingredients | % m/m | film-coated tablet |
| Sodium ibuprofen dihydrate | 52.15 | 512.00 |
| (acetone granulation) | | |
| Copovidone | 8.15 | 80.00 |
| Mannitol | 24.71 | 242.62 |
| Croscarmellose sodium | 4.72 | 46.35 |
| Hydrated colloidal silica | 4.69 | 46.00 |
| Magnesium stearate | 1.73 | 17.00 |
| Poly(vinyl alcohol) composition | 3.85 | 37.76 |
| Total per film-coated tablet | 100.00 | 981.73 |

Its dissolution rate was tested in comparison to the product NUROFEN® FLASH 684 mg lysine salt (equivalent to 400 mg of ibuprofen):

A study of its pharmacokinetics in 20 healthy volunteers after administration of a dose of 512 mg, equivalent to 400 mg of Ibuprofen in acid form, was conducted in comparison to that of a dose of 400 mg of the acid form (NUROFEN® 400 mg).

The results below demonstrate that, advantageously and unexpectedly, the formulation according to the invention (IBUNA) reaches maximum concentration at t=0.62 h (37 min.) as opposed to t=1 h for the reference product (NUROFEN®) corresponding to the acid form of Ibuprofen. The maximum concentrations reached are significantly different, the difference being 30% in favor of the formulation according to the invention.

TABLE 9

| | IBUNA 512 mg | | NUROFEN ® 400 mg | |
|---|---|---|---|---|
| | Batch 715860 | | Batch DL365 | |
| | (N = 20) | | (N = 20) | |
| Parameter (Unit) | Average | (C.V. %) | Average | (C.V. %) |
| Cₘₐₓ (μg/mL) | 47.576 | (21.8) | 34.183 | (21.4) |
| In (Cₘₐₓ) | 3.8401 | (5.6) | 3.5097 | (6.2) |
| Tₘₐₓ (hours)^{a} | 0.62 | (0.33-0.83) | 1.00 | (0.62-4.00) |
| AUC_{0-T} (μg · h/mL) | 123.106 | (25.7) | 128.293 | (24.3) |
| In (AUC_{0-T}) | 4.7821 | (5.3) | 4.8273 | (4.9) |
| AUC_{0-∞} (μg · h/mL) | 126.271 | (26.5) | 132.598 | (24.7) |
| In (AUC_{0-∞}) | 4.8058 | (5.4) | 4.8593 | (5.0) |
| Residual area (%) | 2.33 | (52.3) | 3.13 | (59.0) |
| λ_{Z} (hours⁻¹) | 0.3118 | (15.7) | 0.3025 | (16.4) |
| T_{half} (hours) | 2.27 | (15.1) | 2.36 | (19.3) |

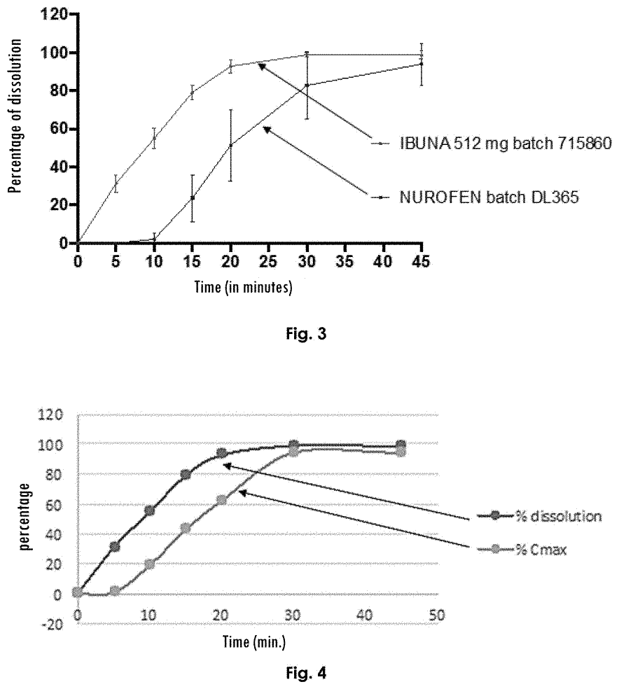

The corresponding curves are shown in FIG. 3.

Moreover, the correlation between the dissolution kinetics results in vitro (pH=6,8) and in vivo was verified (FIG. 4).

Briefly, for the same abscissa (time in min.), the dissolved percentage (% dissolution) and the measured serum concentration expressed as a percentage of measured maximum concentration (% Cₘₐₓ) are reported on the ordinate. After a wait time of 10 minutes, corresponding to the difference in the disintegration times of the tablets according to the invention in vitro and in vivo, we observe a parallelism of the two curves indicating a constant rate of absorption of the active ingredient. This rate is perfectly simulated by the dissolution curve. Beginning at 20 minutes, the 2 curves converge near 100% on the ordinate, reached in 30 minutes.

FIG. 6 shows that in both the film-coated tablets according to the invention and the granules according to the invention, the crystalline structure of the ibuprofen sodium salt dihydrate is maintained. In fact, it is observed that the characteristic peaks of ibuprofen sodium salt dihydrate (from 16° to 21° and at 22° (2-Theta angle)) are retained in each of these products.

### Example 5: Tablets of Ibuprofen Sodium Salt Dihydrate 512 mg+Anhydrous Caffeine 100 mg

Anhydrous caffeine according to the European Pharmacopoeia is preferred, and is added directly to the external phase of the formulation of the ibuprofen sodium salt dihydrate 512 mg tablet.

TABLE 10
IBUCAF - Batch 729740 EXAMPLE 5

| Ingredients | mg/tablet |
|---|---|
| Internal Phase | |
| Ibuprofen sodium salt dihydrate | 512.00 |
| Copovidone (PLASDONE ® S630) | 80.00 |
| Acetone (intermediate solvent) | |
| External phase | |
| Caffeine | 100.00 |
| Mannitol (PEARLITOL ® SD200) | 242.62 |
| Croscarmellose sodium (VIVASOL ®) | 46.35 |
| Hydrated Silica (SYLOID ®) | 46.00 |
| Magnesium stearate | 17.00 |
| Poly(vinyl alcohol) composition | 42.00 |
| Total per film-coated tablet | 1085.97 |
| Mixture before compression | |
| Flow time (g/sec.) | 7.47 |
| Apparent volumes (mL) | |
| V0 | 182 |
| V1250 | 132 |
| Density D0 (g/mL) | 0.55 |
| Compressibility index | 27.5 |
| Hausner ratio | 1.38 |
| Tablets | |
| Average mass (mg) | 1086 |
| Hardness (N) | 118 |
| Disaggregation (min.) | 11:07 |
| Friability % | 0.2 |
| Capping | 0 |
| Binding | 0 |

### Example 6: Capsules of Ibuprofen Sodium Salt Dihydrate 256 and 512 mg

Starting with the granule prepared according to the invention, it is possible to add into the external phase both mannitol to correct variations in the density of the granule and a lubricant, such as stearic acid for example, and directly produce capsules, particularly of size 2, by force filling/trimming.

It is also possible to add mannitol into the external phase without a lubricant, then directly produce capsules, particularly of size 1 or OEL, by force filling/trimming.

### Example 7: Capsules of Ibuprofen Sodium Salt Dihydrate 256 mg and Pseudoephedrine Hydrochloride 30 mg

Alternatively, starting with the granule prepared according to the invention, it is possible to produce a combination with pseudoephedrine hydrochloride, which is added into the external phase along with mannitol and a lubricant. Mannitol is added in variable quantities in order to correct variations in the density of the granule and enable the production of a capsule, particularly of size 1, by force filling/trimming.

1. A method for granulating an amphiphilic active ingredient, or a pharmaceutically acceptable salt thereof, comprising a step for coating the active ingredient in a polar aprotic solvent in the presence of a polymer binder, so as to obtain a granule.

2. The method according to claim 1, wherein the amphiphilic active ingredient comprises at least one carboxylic acid group and at least one aryl group comprising from 6 to 50 carbon atoms.

3. The method according to claim 1, wherein the amphiphilic active ingredient is a nonsteroidal anti-inflammatory drug (NSAID).

4. The method according to claim 1, wherein the amphiphilic active ingredient is an arylacetic NSAID, an arylproprionic NSAID, or an anthranilic NSAID.

5. The method according to claim 1, wherein the amphiphilic active ingredient is selected from the group consisting of ibuprofen, ketoprofen, naproxen, flurbiprofen, oxaprozin, ibufenac, diclofenac, aceclofenac, sulindac, etodolac, ketorolac, indomethacin, mefenamic acid, and niflumic acid.

6. The method according to claim 1, wherein the pharmaceutically acceptable salt of the amphiphilic active ingredient is selected from the group consisting of a lithium salt, a sodium salt, a potassium salt, a calcium salt, an aluminum salt, a magnesium salt, a zinc salt, an arginine salt, a lysine salt, a histidine salt, a choline salt, an ethanolamine salt, a diethanolamine salt, a triethanolamine salt, an ethylenediamine salt, and a meglumine salt.

7. The method according to claim 1, wherein the amphiphilic active ingredient or the pharmaceutically acceptable salt thereof is the lysine salt or the sodium salt of ibuprofen.

8. The method according to claim 1, wherein the polymer binder is a polyvinylpyrrolidone (povidone) or a polyvinylpyrrolidone copolymer, in particular copovidone (a copolymer of polyvinylpyrrolidone and vinyl acetate), a polyethylene glycol (PEG), a polyoxypropylene copolymer, or a methacrylate copolymer.

9. The method according to claim 1, wherein the quantity of polymer binder is of at least 5% by weight relative to the weight of active ingredient.

10. The method according to claim 1, wherein the polar aprotic solvent is selected from the group consisting of acetone, ethyl acetate, acetonitrile, and N,N-dimethylformamide, or a mixture thereof.

11. The method according to claim 1, further comprising a step of drying and/or sieving the granule.

12. A granule that can be obtained by the method according to claim 1.

13. A granule comprising a polar active ingredient, or a pharmaceutically acceptable salt thereof, coated with a polymer binder and having a density of 0.5 to 0.7 g/mL and/or a flow rate of 3 to 15 g/sec.

14. The granule according to claim 13, wherein the amphiphilic active ingredient comprises at least one carboxylic acid group and at least one aryl group comprising from 6 to 50 carbon atoms.

15. The granule according to claim 13, wherein the amphiphilic active ingredient is a nonsteroidal anti-inflammatory drug (NSAID).

16. The granule according to claim 13, wherein the amphiphilic active ingredient is an arylacetic NSAID, an arylproprionic NSAID, or an anthranilic NSAID.

17. The granule according to claim 13, wherein the polar active ingredient is selected from the group consisting of ibuprofen, ketoprofen, naproxen, flurbiprofen, oxaprozin, ibufenac, diclofenac, aceclofenac, sulindac, etodolac, ketorolac, indomethacin, mefenamic acid, and niflumic acid.

18. The granule according to claim 13, wherein the pharmaceutically acceptable salt of the polar active ingredient is selected from the group consisting of a lithium salt, a sodium salt, a potassium salt, a calcium salt, an aluminum salt, a magnesium salt, a zinc salt, an arginine salt, a lysine salt, a histidine salt, a choline salt, an ethanolamine salt, a diethanolamine salt, a triethanolamine salt, an ethylenediamine salt, and a meglumine salt.

19. The granule according to claim 13, wherein the amphiphilic active ingredient or the pharmaceutically acceptable salt thereof is the lysine salt or the sodium salt of ibuprofen.

20. The granule according to claim 13, wherein the polymer binder is a polyvinylpyrrolidone (povidone) or a polyvinylpyrrolidone copolymer, in particular copovidone (a copolymer of polyvinylpyrrolidone and vinyl acetate), a polyethylene glycol (PEG), a polyoxypropylene copolymer, or a methacrylate copolymer.

21. The granule according to claim 13, wherein the quantity of polymer binder is of at least 5% by weight relative to the weight of active ingredient.

22. A pharmaceutical composition or medicine comprising a granule according to claim 12 and at least one pharmaceutically acceptable vehicle or excipient.

23. (canceled)

24. (canceled)

25. (canceled)

26. (canceled)

27. (canceled)

28. A method for the prevention or treatment of pain, fever, and/or inflammation in an in an individual in need thereof, comprising administering to the individual a therapeutically effective amount at least one granule according to claim 12.
